(19) United States
(12) Patent Application Publication
(10) Pub. No.: US 2022/0042070 A1
(43) Pub. Date: Feb. 10, 2022

(54) METHODS AND SYSTEMS FOR READING DNA STORAGE GENES

(71) Applicant: Seagate Technology LLC, Fremont, CA (US)

(72) Inventors: Walter R. EPPLER, Cranberry Township, PA (US); Gemma MENDONSA, EDINA, MN (US)

(21) Appl. No.: 16/985,117

(22) Filed: Aug. 4, 2020

(51) Int. Cl. C12Q 1/6806; G06N 3/12; G16B 50/00

(52) U.S. Cl. CPC C12Q 1/6806; G16B 50/00; G06N 3/123

(57) ABSTRACT

Methods and systems for use in reading DNA storage genes generally include removing one or more linking symbols from a first strand of a DNA storage gene, introducing a test symbol pool to the DNA storage gene, and replacing a data symbol in the first strand of the DNA storage gene with a single stranded test symbol from the test symbol pool when the linking symbol and data symbol of the single stranded test symbol are complimentary to an adjacent linking symbol and data symbol in the second strand of the DNA storage gene. The DNA storage gene is then scanned to identify locations on the DNA storage gene where the linking symbol is double stranded or single stranded. The locations where double stranded linking symbols are detected are then used, along with the composition of the test symbol pool, to read the DNA storage gene.

### SUMMARY

This summary is provided to introduce a selection of concepts in a simplified form that are further described below in the Detailed Description. This Summary is not intended to identify key features or essential features of the claimed subject matter, nor is it intended to be used to limit the scope of the claimed subject matter. Other features, details, utilities, and advantages of the claimed subject matter will be apparent from the following, more particular written Detailed Description of various implementations as further illustrated in the accompanying drawings and defined in the appended claims.

Various embodiments of methods and systems for reading DNA storage genes are described herein. In some embodiments, a method for use in reading a DNA storage gene includes removing one or more linking symbols from a first strand of a DNA storage gene and introducing a test symbol pool to the DNA storage gene. The test symbol pool can include a plurality of single stranded test symbols, each single stranded test symbol comprising a data symbol and a linking symbol. The method further includes replacing a data symbol in the first strand of the DNA storage gene with a single stranded test symbol from the test symbol pool when the data symbol and the linking symbol in the single stranded test symbol are complimentary, respectively, to an adjacent linking symbol and data symbol in the second strand of the DNA storage gene. The method further includes scanning the DNA storage gene to identify whether each linking symbol in the DNA storage gene is single stranded DNA or double stranded DNA, and recording the locations where the linking symbol is double stranded DNA.

In some embodiments, a method for use in reading a DNA storage gene includes providing a first DNA storage gene having one or more linking symbols removed from a first strand of the DNA storage gene and introducing a first test symbol pool to the first DNA storage gene. The first test symbol pool includes a plurality of single stranded test symbols, each single stranded test symbol including one of a first set of data symbols and one of a first set of linking symbols. The method further includes replacing a data symbol in the first strand of the first DNA storage gene with a single stranded test symbol from the first test symbol pool when the data symbol and the linking symbol in the single stranded test symbol are complimentary, respectively, to an adjacent linking symbol and data symbol in the second strand of the first DNA storage gene. The method further incudes scanning the first DNA storage gene to identify whether each linking symbol in the first DNA storage gene is single stranded DNA or double stranded DNA and recording each location on the first DNA storage gene where the linking symbol is double stranded DNA. The method further includes providing a second DNA storage gene having one or more linking symbols removed from a first strand of the second DNA storage gene, the second DNA storage gene being identical to the first DNA storage gene, and introducing a second test symbol pool to the second DNA storage gene. The second test symbol pool includes a plurality of single stranded test symbols, each single stranded test symbol including one of a second set of data symbols and one of the first set of linking symbols, the second set of data symbols being different from the first set of data symbols. The method further includes replacing a data symbol in the second strand of the second DNA storage gene with a single stranded test symbol from the second test symbol pool when the data symbol and the linking symbol in the single stranded test symbol are complimentary, respectively, to an adjacent linking symbol and data symbol in the second strand of the second DNA storage gene. The method further includes scanning the second DNA storage gene to identify whether each linking symbol in the second DNA storage gene is single stranded DNA or double stranded DNA and recording each location on the second DNA storage gene where the linking symbol is double stranded DNA. The method further includes using the recoded linking symbol locations and the composition of the first test symbol pool and the second test symbol pool to read the DNA storage gene.

In some embodiments, a system for use in reading a DNA storage gene includes a reaction vessel, an enzyme source, one or more test symbol pool sources, and a scanner. The reaction vessel is configured to receive a DNA storage gene. The enzyme source is in fluid communication with the reaction vessel and stores an enzyme source that is configured to remove one or more linking symbols from a first strand of a DNA storage gene. The one or more test symbol pool sources are in fluid communication with the reaction vessel and store a test symbol pool including a plurality of single stranded test symbols, each single stranded test symbol including a data symbol and a linking symbol. The scanner is configured to scan a DNA storage gene located in the reaction vessel and distinguish between single stranded DNA and double stranded DNA in the DNA storage gene, detect a single stranded DNA overhang, or both.

### BRIEF DESCRIPTION OF THE DRAWINGS

A further understanding of the nature and advantages of the present technology may be realized by reference to the figures, which are described in the remaining portion of the specification.

FIG. 1 illustrates a flow chart of a method for use in reading DNA storage genes, according to one aspect of the present disclosure.

FIGS. 2A-2D present a schematic illustration of a method for use in reading DNA storage genes, according to one aspect of the present disclosure.

FIG. 3 illustrate a flow chart of a method for use in reading DNA storage genes, according to one aspect of the present disclosure.

FIGS. 4A and 4B illustrate a test symbol pool protocol for use in reading DNA storage genes, according to one aspect of the present disclosure.

FIG. 5 presents a schematic illustration of a system for use in reading DNA storage genes, according to one aspect of the present disclosure.

### DETAILED DESCRIPTION

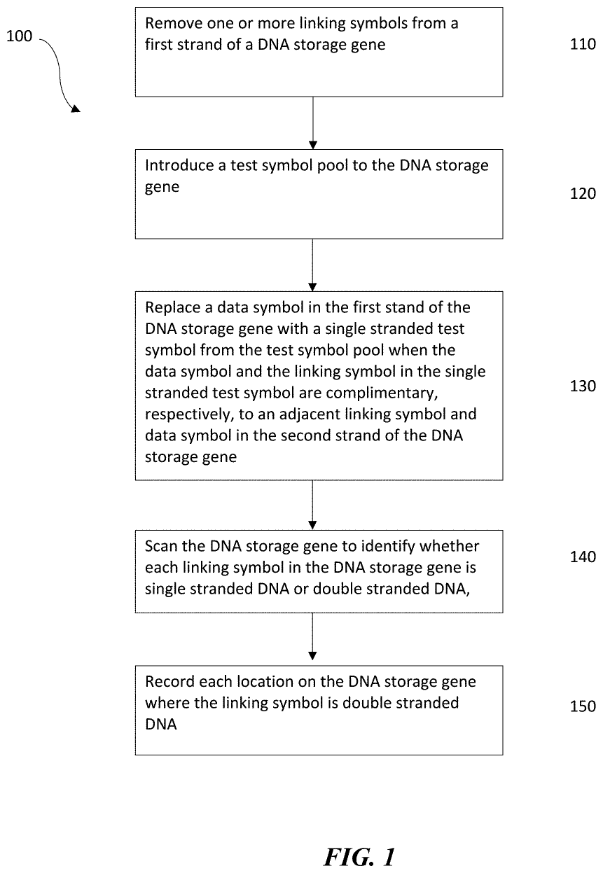

With reference to FIG. 1, one embodiment of a method 100 for use in reading a DNA storage gene generally comprises a step 110 of removing one or more linking symbols from a first strand of a DNA storage gene, a step 120 of introducing a test symbol pool to the DNA storage gene, a step 130 of replacing a data symbol in the first stand of the DNA storage gene with a single stranded test symbol from the test symbol pool when the data symbol and the linking symbol in the single stranded test symbol are complimentary, respectively, to an adjacent linking symbol and data symbol in the second strand of the DNA storage gene, a step 140 of scanning the DNA storage gene to identify whether each linking symbol in the DNA storage gene is single stranded DNA or double stranded DNA, and a step 150 of recording each location on the DNA storage gene where the linking symbol is double stranded DNA.

Regarding step 110, a DNA storage gene is provided and then manipulated in order to remove one or more linking symbols from a first strand of the DNA storage gene. Any suitable DNA storage gene can be used in step 110 and as part of the overall method 100 of reading a DNA storage gene. DNA storage genes serve as volumetrically efficient archival storage mediums by way of using an encoding scheme to construct/synthesize a sequence of base pairs, after which the base pairs can be decoded/read in order to communicate information via the DNA storage gene.

In some embodiments, the DNA storage gene is a long strand of double stranded DNA made up of multiple individually selected data symbols assembled in a specific order using a predefined set of linking symbols. The data symbols themselves each comprise a unique sequence of one or more base pairs. The DNA storage gene may comprise alternating fixed length sections of data symbols and linking symbols. The DNA storage gene may further comprise binding symbols used to connect a linking symbol to either end of the data symbol. In some embodiments, the linking symbol prior to the data symbol when reviewing the DNA storage gene from left to right is the linking symbol associated with the data symbol, while the linking symbol following the data symbol when reviewing the DNA storage gene from left to right is the linking symbol associated with the next data symbol in the DNA storage gene. Thus, using the nomenclature where DS stands for data symbol, LS stands for linking symbol, and BS-L and BS—R stand for left binding symbol and right binding symbol, respectively, the DNA storage gene may have a construction as follows:

BS—R:LS1:BS-L:DS1:BS—R:LS2:BS-L:DS2:BS—R:LS1:BS-L:DS3:BS—R:LS2:BS-L

The linking symbols used in DNA storage gene may be selected from a full set of known linking symbols that arrange in repeating order. In this manner, linking symbols may be reused in the DNA storage gene such that the DNA storage gene need not use a unique linking symbol prior to every data symbol, but the linking symbols will arrange in repeating order. Thus, in the representation given above, two linking symbols are used (LS1 and LS2), and the pattern of linking symbols used in the DNA storage gene repeats LS1, LS2, LS1, LS2 . . . , though with different, random data symbols attached to the linking symbols.

The units of associated linking symbols and data symbols can be referred to as a data block, and the DNA storage gene is designed such that the data blocks assemble in sequence, using the linking symbol sequence as the overall sequencing guide. When the sequence of available linking symbols ends, the sequence repeats in order to extend the length of the DNA storage gene. The binding symbols may be constant, such that the same left and right binding symbols are used in each data block.

With reference again to step 110, the DNA storage gene provided for reading is manipulated such that one or more linking symbols on a first strand of the double stranded DNA storage gene are removed from the DNA storage gene. The result of step 110 is a DNA storage gene having various segments that are single stranded due to the removal of a linking symbols from the first strand of the double stranded DNA storage gene. In some embodiments, step 110 is carried out such that all linking symbols from a first strand of the DNA storage gene are removed, thereby providing a DNA storage gene that is single stranded at all linking symbol locations.

Any manner of removing the linking symbols from a first strand of the DNA storage gene can be used as part of step 110. In some embodiments, the linking symbols are removed from the first strand of the DNA storage gene by nicking or cutting the first strand of the DNA storage gene at the juncture of each linking symbol and the adjacent binding symbols. Referring again to the DNA storage gene construction described previously, the first strand of the DNA storage gene can be nicked or cut at the juncture of LS1 and BS-L, the juncture of LS1 and BS—R, the juncture of LS2 and BS-L, the juncture of LS2 and BS—R, and so on.

Any manner of creating these links or cuts in the linking symbols can be used. In some embodiments, an enzyme is programmed to make these cuts such that the introduction of the enzyme to the DNA storage gene results in the cuts being made at either end of the linking symbols. Once cut, further steps may be used to remove the linking symbols from the first strand of the DNA symbol. In some embodiments, a heating step is used in order to remove the cut linking symbols from the first strand of the DNA storage gene. As noted previously, the result of this step is to provide a DNA storage gene that is single stranded at one or more linking symbol locations. The removal of the one or more linking symbols also results in the creation of a toe-hold in the first strand of the DNA storage gene. These toe-holds can be used in subsequent steps described in greater detail below where complimentary single stranded test symbols replace data symbols in the first strand of the DNA storage gene and thereby revert the single stranded linking symbols to double stranded linking symbols.

In step 120, a test symbol pool is introduced to the DNA storage gene having one or more single stranded test symbols. The test symbol pool is comprised of a plurality of single stranded test symbols, each test symbol comprising a data symbol selected from a first set of data symbols and a linking symbol selected from a first set of linking symbols. Each single stranded test symbol may also include a right binding signal and a left binding signal. Each single stranded test symbol may further include an anchor symbol (AS), which may be located at a first end of the test symbol, and with the linking symbol being located at the opposite (second) end of the test symbol. In some embodiments, the single stranded test symbols that make up the test symbol pool may have the following construction:

LSx:BS-L:DSx:BS—R:AS

The test symbol pool is designed to include single stranded test symbols of every possible combination of the first set of data symbols and the first set of linking symbols used to make up the test symbol pool. Thus, in an embodiment where the test symbol pool includes a first set of data symbols DS1, DS2, DS3 and DS4, and a first set of linking symbols L1 and L2, the test symbol pool includes single stranded test symbols of L1-DS1, L1-DS2, L1-DS3, L1-DS4, L2-DS1, L2-DS2, L2-DS3 and L2-DS4. The first set of data symbols and the first set of linking symbols will include data symbols and linking symbols that are present in the DNA storage gene. In some embodiments, the first set of data symbols used in the test symbol pool may include less than all of the data symbols used in the DNA storage gene, while the first set of linking symbols used in the test symbol pool will include any and all of the linking symbols used in the DNA storage gene.

In step 120, the test symbol pool is introduced to the DNA storage gene in any manner that allows for interaction between the test symbol pool and the DNA storage gene, such as the replacement of a data symbol in the first strand of the DNA storage gene with a single stranded test symbol from the test symbol pool. In step 130, this replacement occurs at any location on the DNA storage gene where a single stranded test symbol, comprising a data symbol and a linking symbol, is complimentary to an adjacent data symbol and linking symbol in the second strand of the DNA storage gene. In this replacement, a data symbol in the first strand of the DNA storage gene, which may include a right binding symbol and a left binding symbol, is removed from the first strand. In its place, a test symbol from the test symbol pool that is comprised of a data symbol and linking symbol that is complimentary to the data symbol and linking symbol in the second strand of the DNA storage gene at the location where the data symbol from the first strand previously resided attaches to the first strand of the DNA storage gene. Because the test symbol includes a data symbol and a linking symbol, the replacement of the data symbol in the first strand with the test symbol results in that linking symbol location of the DNA storage gene reverting back to double stranded DNA. This replacement occurs in any location where a test symbol exists in the test symbol pool that is complimentary to a linking symbol/data symbol pair in the second strand of the DNA storage gene.

In locations along the DNA storage gene where the test symbol pool does not include a single stranded test symbol that compliments a linking symbol/data symbol pair in the second strand of the DNA storage symbol, no replacement takes place. As such, the first strand of the DNA storage gene in those locations retains the original data symbol and continues to be without the linking symbol removed from the first strand of the DNA storage gene in step 110. In some embodiments, the DNA storage gene following step 130 will include some locations where the linking symbol location has reverted to double strand DNA and some locations where the linking symbol locations remain single stranded. Depending on the composition of the test symbol pool and the sequence of data symbols in the DNA storage gene, it is also possible that following step 130, all linking symbol locations on the DNA storage gene are reverted back to double stranded DNA or all linking symbol locations on the DNA storage gene remain single stranded DNA.

The specific manner in which the test symbol replaces the data symbol in the first strand of the DNA symbol when there is a compliment between the test symbol and the linking symbol/data symbol pair in the second strand of the DNA storage gene is not limited. In some embodiments, the mechanism for replacement is a toe-hold mediated strand displacement (TMSD) reaction.

Any manner of preparing the test symbol pool can be used, provided that the test symbol pool includes test symbols for every combination of the first set of test symbols and the first set of linking symbols selected for a given test symbol pool. In some embodiments, the single stranded test symbols are created from double stranded versions of the test symbols that are denatured, annealed to a methanol-responsive polymer anchor or magnetic particle anchor for capture, and then, following pull down and release from the polymer anchor, are refined to a single stranded test symbols. In embodiments where the test symbols include an anchor symbol but where the anchor symbol interferes with toe-hold mediated strand displacement or is otherwise not helpful during subsequent scanning of the DNA storage gene, the anchor may be removed from the test symbols as part of preparing the test symbols that will make up the test symbol pool.

As discussed in greater detail below, the overall method 100 used for reading DNA storage gene may use multiple test symbol pools, wherein the composition of each test symbol pool is different. In some embodiments, each test symbol pool includes a different set of data symbols. A different set of data symbols means that each set of data symbols is different from the other set of data symbols by at least one data symbol. Thus, some sets of data symbols with have common data symbols, but will not be completely overlapping. In some embodiments, a first set of data symbols used for a first test symbol pool may include DS1, DS2, DS3 and DS4, while a second set of data symbols used for a second test symbol pool may include DS1, DS2, and DS3. Thus, while both sets of data symbols include DS1, DS2 and DS3, the sets of data symbols are considered different because of the presence of DS4 in the first set of data symbols and the absence of DS4 in the second set of data symbols. The set of linking symbols between different test symbol pools remains the same.

In step 140, the DNA storage gene is scanned to identify whether each linking symbol in the DNA storage gene is single stranded DNA or double stranded DNA. The locations where a linking symbol remains single stranded following step 130 allows for the inference that the test symbol pool used in step 130 did not include a test symbol including the data symbol at the location on the DNA storage gene where the linking symbol remains single stranded. Similarly, the locations where a linking symbol has reverted back to double stranded following step 130 allows for the inference that the test symbol pool used in step 130 did include a test symbol including the data symbol at the location on the DNA storage gene where the linking symbol has reverted back to double stranded. As discussed in greater detail below, this information along with the known composition of the test symbol pool used, can be used to determine the specific data symbol at each location along the DNA storage gene and thereby permit reading of the DNA storage gene. Any manner of scanning the DNA storage gene to identify double stranded or single stranded locations can be used. In some embodiments, a scanning device capable of distinguishing between double stranded DNA and single stranded DNA is used to carry out step 140.

In other embodiments, step 140 includes scanning the DNA storage gene to identify anchor tails of the test symbols that have replaced data symbols in the first strand of the DNA storage gene. Similar to detecting double stranded locations, this method allows for similar inferences to those detailed above, where the presence of an anchor tail means a replacement has taken place and therefore the test symbol pool included a test symbol having a data symbol also included in the DNA storage gene. Any device capable of identifying the presence of such anchor tails can be used to carrying out this version of step 140. In some embodiments, the anchor tails are magnetic particle anchors, which can be detected through the use of magnetic sensors

In step 150, the location on the DNA storage gene where double stranded linking symbols are located (or where anchor symbols are identified) is recorded. Any manner of recording this information can be used, such as through the use of an associated computer system/processor used as part of carrying out the method 100. A computer system/processor may be in direct or indirect communication with the scanner used in step 140 so that location information is relayed from the scanner to the computer system/processor used to record location information. As discussed in greater detail below, this location information, along with information relating the composition of the test symbol pool, can be used as part of reading/decoding the DNA storage gene.

In some embodiments, the method 100 is carried out multiple times, with each iteration of the method 100 being carried out using a test symbol pool with a different composition of test symbols. In some embodiments, the method 100 is carried out concurrently using different test symbol pools in order to expedite the overall process of reading the DNA storage gene. When location data is recorded from multiple methods 100 performed serially or in parallel and each method 100 using a test symbol pool of differing test symbol compositions, relatively basic logic can be used to identify the data symbol at each location of the DNA storage gene and thereby read the DNA storage gene.

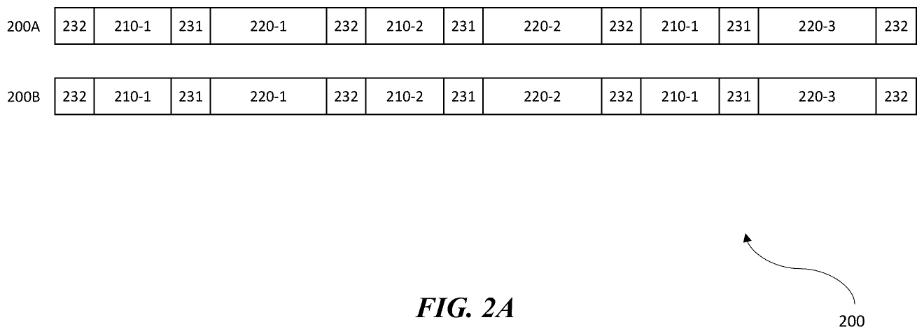

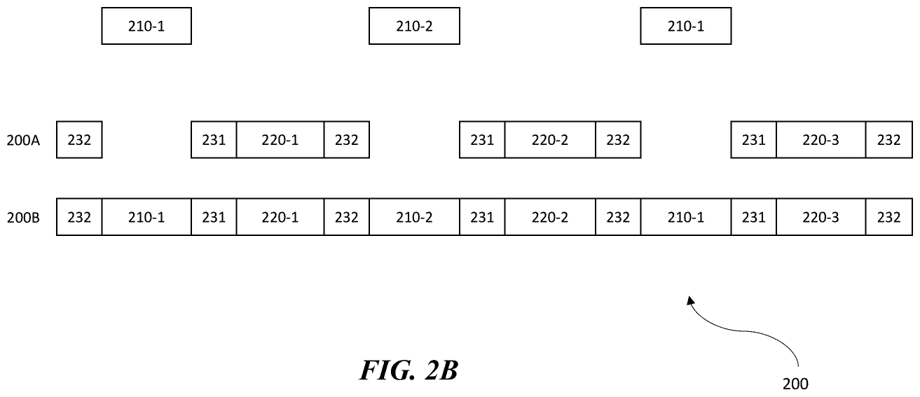

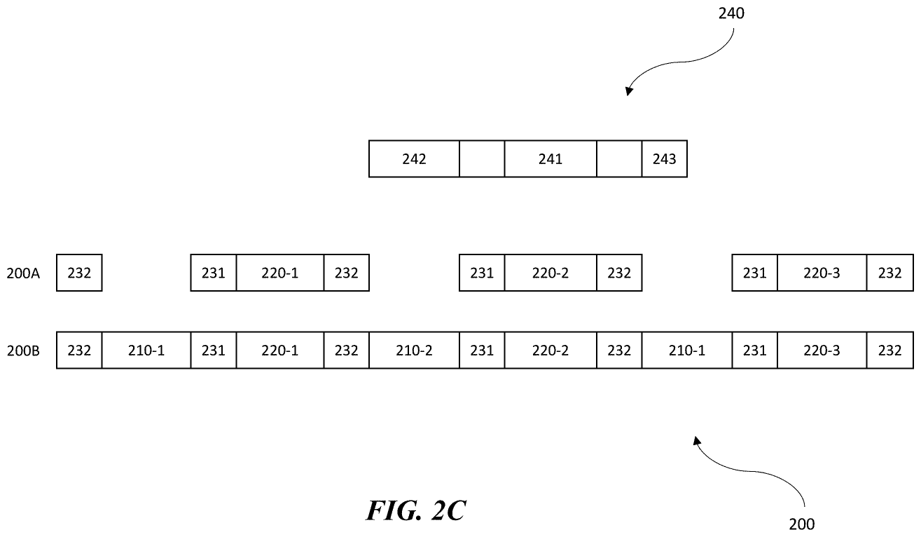

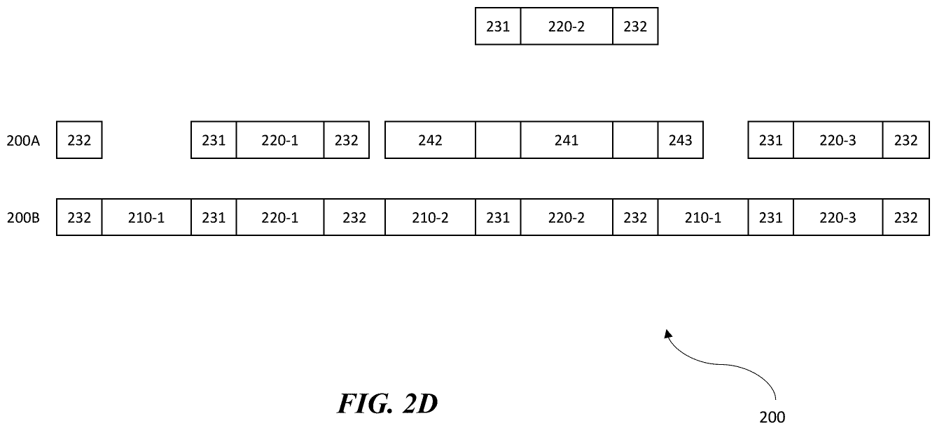

With reference to FIGS. 2A-2D, a schematic illustration of steps 110, 120 and 130 of FIG. 1 is shown. FIG. 2A generally illustrates a DNA storage gene 200 suitable for use in the methods described herein, the DNA storage gene 200 generally including a first strand 200A and a second strand 200B, and each strand 200A, 200B generally including a plurality of linking symbols 210-1, 210-2, a plurality of data symbols 220-1, 220-2, 220-3, etc., and left binding symbols 231 and right binding symbols 232 on either end of each of the data symbols 220-1, 220-2, 220-3, etc. As discussed previously, the data symbols 220-1, 220-2, 220-3, etc., are comprised of sequences of one or more base pairs. The DNA storage gene 200 may include any number of different data symbols, and any data symbol may be repeated one or more times in the same DNA storage gene. As also discussed previously, the linking symbols are selected from a defined set of linking symbols and appear in the DNA storage gene 200 in a repeating sequence.

FIG. 2B generally correlates to step 110 of FIG. 1 wherein the DNA storage gene 200 is manipulated to remove one or more linking symbols 210-1, 210-2 from a first strand 200A of the DNA storage gene 200. In some embodiments, all linking symbols present in the DNA storage gene 200 are removed from the first strand 200A, such as through the use of a programmed enzyme that nicks either end of each linking symbol 210-1, 210-2 followed by heating to remove the linking symbols 210-1, 210-2 from the first strand 200A.

FIG. 2C generally correlates to step 120 of FIG. 1, wherein a plurality of single stranded test symbols 240 that are part of a test symbol pool are introduced to the DNA storage gene 200. For simplicity sake, FIG. 2C shows only a single test symbol 240 introduced to the DNA storage gene 200, but it should be appreciated that the test symbol pool will typically include a plurality of many different test symbols. Test symbol 240 generally includes a data symbol 241, a linking symbol 242 at one end of the test symbol 240 and an anchor symbol 243 at an opposite end of the test symbol 240. The data symbols and linking symbols that make up the different test symbols 240 that are part of the test symbol pool introduced to the DNA storage gene 200 are selected from the same data symbols and linking symbols present in the DNA storage gene 200.

FIG. 2D generally correlates to step 130 of FIG. 1, wherein the test symbol 240 replaces data symbol 220-2 in the first strand 200A, such as via toe-hold mediated strand displacement. Symbol 240 replaces data symbol 220-2 in first strand 200A because the data symbol 241 and linking symbol 242 of the test symbol 240 are complimentary to the data symbol and linking symbol in the second strand 200A at the location of the data symbol 220-2. Due to the replacement of the data symbol 220-2 with the test symbol 240, which includes both data symbol 241 and linking symbol 242, the portion of the DNA storage gene 200 at the previous location of data symbol 220-2 is now double stranded, including at the linking symbol. As also shown in FIG. 2D, no replacement of data symbol 220-1 or 220-3 takes place because the data symbol 241 and linking symbol 242 of the test symbol 240 are not complimentary to the data symbol and linking symbol in the second strand 200A at the location of either of the data symbol 220-1 or 220-3. As such, the DNA storage gene 200 remains single stranded at the linking symbol 210-1 adjacent each of data symbol 220-1 and 220-3.

Following the replacement shown in FIG. 2D, the DNA storage gene 200 can be scanned in order to identify which linking symbol portions of the DNA storage gene 200 are double stranded and single stranded. The double stranded locations are recorded and when used in connection with the known composition of the test symbol pool and potentially other location data recorded upon repeating the process with a test symbol pool of a different composition, the DNA storage gene can be read.

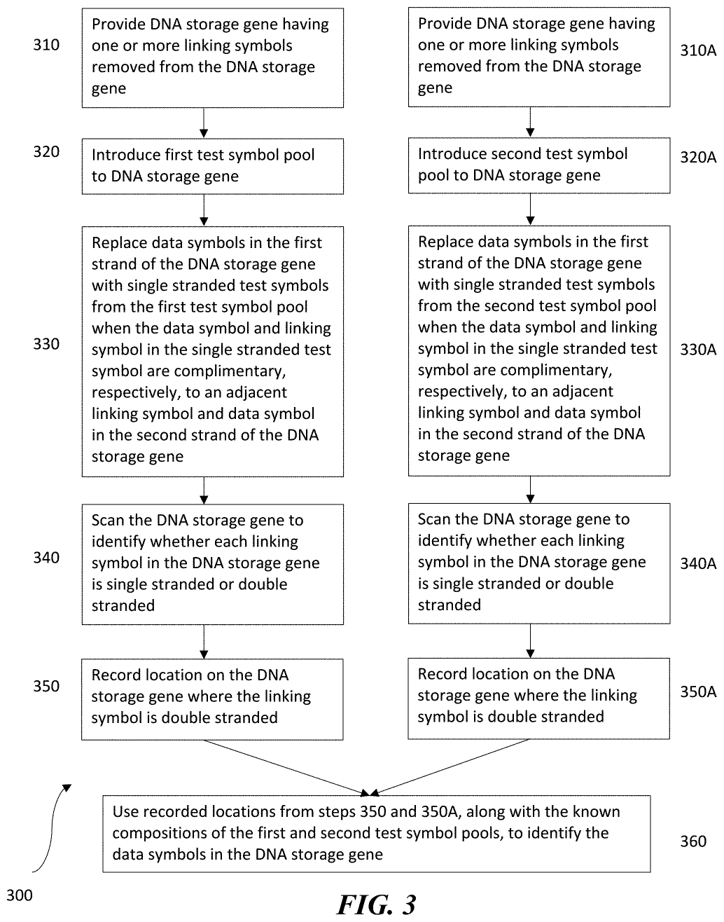

With reference to FIG. 3, an embodiment of a method 300 for use in reading a DNA storage gene is similar in some respects to the method 100 shown in FIG. 1, but with the method 300 showing the manner in which iterations of the method are carried out in parallel in order to expedite the overall process of reading the DNA storage gene. Parallel iterations of the method 300 generally use the same DNA storage gene modified to remove linking symbols in a first strand of the DNA storage gene, but generally use different test symbol pools to extract additional information regarding the content of the DNA storage gene that when used together at the end of the iterations can be used to read the DNA storage gene.

The method 300 general includes step 310 of providing a first DNA storage gene having one or more linking symbols removed from the first DNA storage gene. In some embodiments, all linking symbols are removed from the first strand of the first DNA storage gene. The specific manner and details of removing linking symbols from the first DNA storage gene is similar or identical to step 110 described in greater detail above.

In step 320, a first test symbol pool is introduced to the DNA storage gene. The first test symbol pool includes a plurality of single stranded test symbols, each single stranded test symbol comprising one data symbol from a first set of data symbols and one linking symbol from a first set of linking symbols. In some embodiments, the test symbol pool includes single stranded test symbols for every combination of the first set of data symbols and the first set of data symbols. Furthermore, the data symbols in the first set of data symbols and the linking symbols in the first set of linking symbols include only data symbols and linking symbols present in the DNA storage gene. The specific manner and details of introducing the first test symbol pool to the DNA storage gene can be similar or identical to step 120 described in greater detail above.

In step 330, data symbols in the first strand of the DNA storage gene are replaced with single stranded test symbols from the first test symbol pool when the data symbol and linking symbol in the single stranded test symbol are complimentary, respectively, to an adjacent linking symbol and data symbol in the second strand of the DNA storage gene. Where these replacements take place, the linking symbol portion of the DNA storage gene reverts back to double stranded DNA. The replacement of a data symbol in the first strand of the DNA storage gene with a single stranded test symbol may be carried out via TMSD. The specific manner and details of replacing the data symbols in the first strand of the DNA storage gene with single stranded test symbols from the first test symbol pool can be similar or identical to step 130 described in greater detail above.

In step 340, the DNA storage gene is scanned to identify whether each linking symbol in the DNA storage gene is single stranded or double stranded. The presence of a double stranded linking symbol in the DNA storage gene infers that the test symbol pool included a data symbol present in the DNA storage gene. The specific manner and details of scanning the DNA storage gene can be similar or identical to step 140 described in greater detail above.

In step 350, the location on the DNA storage gene where the linking symbol is double stranded is recorded. This recorded location information is associated with the known composition of the test symbol pool used in step 320 so that it can later be used to read the DNA storage gene. The specific manner and details of recording the location information can be similar or identical to step 150 described in greater detail above.

Method 300 further includes steps 310A, 320A, 330A, 340A and 350A, which are essentially identical to steps 310, 320, 330, 340 and 350, respectively, but which use a second DNA storage gene and a second test symbol pool. In some embodiments, the second DNA storage gene is identical to the first DNA storage gene, including the removal of linking symbols from the same locations as in the first DNA storage gene (in embodiments where the not all linking symbols are removed from the first DNA storage gene). The second test symbol pool introduced in step 320A has a different composition of test symbols from the composition of test symbols included in first test symbol pool of step 320. Generally speaking, the second test symbol pool comprises test symbols made up of data symbols from a second set of data symbols and linking symbols from the same first set of linking symbols used for the first test symbol pool. The second set of data symbols is different from the first set of data symbols in that the second data symbol includes at least one data symbol present in the first set of data symbols or excludes at least one data symbol present in the first set of data symbols. Thus, while the first set of data symbols and the second set of data symbols may include the same data symbols, the data symbols of each set of data symbols are not perfectly identical.

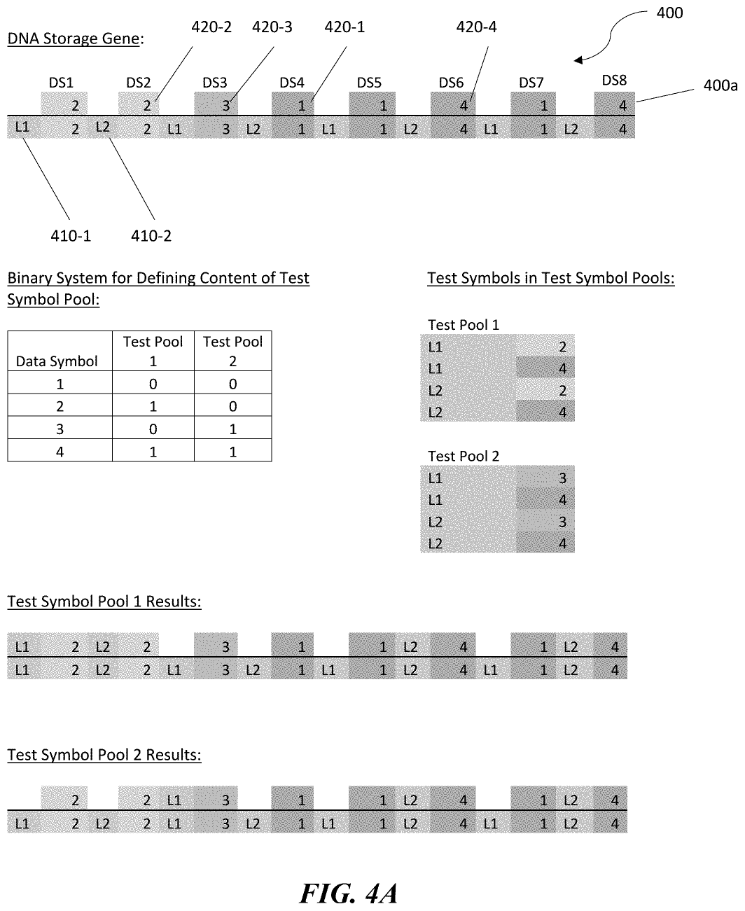

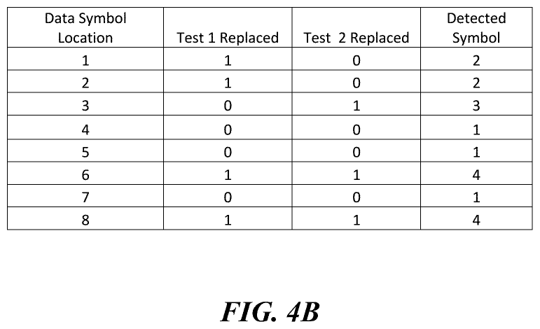

In step 360, the recorded locations from steps 350 and 350A, along with the known compositions of the first and second test symbol pools, are used to identify the data symbols, including their sequence, in the DNA storage gene. Relatively simple logic can be used to read the DNA storage gene based on this information and as described in greater detail below with respect to FIG. 4.

FIGS. 4A and 4B illustrate an embodiment of a test symbol pool protocol for use in reading DNA storage genes. In the embodiment illustrated in FIG. 4A, a DNA storage gene 400 having all linking symbols removed from the first strand 400a of the storage gene 400 is provided. It is known that the storage gene 400 uses linking symbols 410-1 and 410-2 and data symbols 420-1, 420-2, 420-3, and 420-4, and that the DNA storage gene 400 includes eight data symbol locations (DS1, DS2, DS3 . . . DS8). While FIG. 4A shows the specific data symbols and order of data symbols that make up the DNA storage gene 400, it is assumed that in a DNA storage gene reading scenario, only the linking symbols and data symbols that may be present in the DNA storage gene are known, but not the specific data symbol at each data symbol location.

In order to determine the data symbol at each data symbol location DS1-DS8 of the DNA storage gene 400, a protocol is introduced where different test symbol pools are introduced to the DNA storage gene. A binary system may be used to determine the composition of each test symbol pool used, where ones and zeros denote the presence or absence, respectively, of a possible data symbol present in the DNA storage gene 400. Thus, as shown in FIG. 4, the binary system is used to define a first test symbol pool that includes data symbols 420-2 and 420-4 (denoted by ones for these data symbols) and excludes data symbols 420-1 and 420-3 (denoted by zeroes for these data symbols). A second test symbol pool includes data symbols 420-3 and 420 4 (denoted by ones for these data symbols) and excludes data symbols 420-1 and 420-2 (denoted by zeroes for these data symbols).

FIG. 4A further illustrates the composition of test symbols included in these two test symbol pools based on the binary symbol protocol discussed above, wherein every linking symbol used in the DNA storage gene is paired with every data symbol present in the test symbol pool to form the test symbols. As noted previously, the DNA storage gene 400 is known to include linking symbols 410-1 and 420-2 in repeating sequence along the length of the DNA storage gene. Thus, the first test symbol pool must include test symbols of linking symbol 410-1 paired with data symbol 420-2, linking symbol 410-2 paired with data symbol 420-2, linking symbol 410-1 paired with data symbol 420-4 and linking symbol 410-2 paired with data symbol 420-4. Similarly, second test symbol pool must include test symbols of linking symbol 410-1 paired with data symbol 420-3, linking symbol 410-2 paired with data symbol 420-3, linking symbol 410-1 paired with data symbol 420-4 and linking symbol 410-2 paired with data symbol 420-4.

FIG. 4A next shows the results from introducing the first test symbol pool to the DNA storage gene 400, and more specifically, where replacements of data symbols with test symbols occurred to thereby provide a double stranded linking symbol at the position proceeding each data symbol location DS1-DS8. The results of introducing the first test symbol pool to the DNA storage gene 400 shows that double stranded linking symbols exist prior to DS1, DS2, DS6 and DS8. These locations are recorded and maintained associated with the composition of the first test symbol pool (which included data symbols 420-2 and 420-4).

Because only limited conclusions can be drawn from this information as it relates to identifying the data symbol at each data symbol location, the second test symbol pool is introduced to the DNA storage gene having all linking symbols removed. Generally, the DNA storage gene used with the second test symbol pool will be a fresh version of the DNA storage gene in that no replacements have yet taken place on the DNA storage gene to which the second test symbol pool is introduced. FIG. 4A shows the results from introducing from introducing the second test symbol pool to the DNA storage gene 400, and more specifically, where replacements of data symbols with test symbols occurred to thereby provide a double stranded linking symbol at the position proceeding each data symbol location DS1-DS8. The results of introducing the second test symbol pool to the DNA storage gene 400 shows that double stranded linking symbols exist prior to DS3, DS6 and DS8. These locations are recorded and maintained associated with the composition of the second test symbol pool (which included data symbols 420-3 and 420-4).

The combination of the data collected from the first test symbol pool run and the second test symbol pool can now be used together to identify the specific data symbol of each data symbol location in the DNA storage gene. FIG. 4B provides the aggregated data and how it can be used to identify the data symbol at each location. Basic logic and process of elimination is used to make the identifications. With respect to DS1, it is known that a replacement occurred at DS1 when the first test symbol pool was introduced, meaning that the data symbol at DS1 must be either data symbol 420-2 or 420-4. It is also known that no replacement took place when the second test symbol pool was introduced, meaning that the data symbol at DS1 cannot be data symbol 420-3 or 420-4. Thus, by logic and process of elimination, DS1 must be data symbol 420-2 by virtue of the information obtained from the first test symbol pool run and the second test symbol pool run. With respect to DS2, identical results to DS1 were obtained from the two test symbol pool runs, meaning DS2 must also be data symbol 420-2. With respect to DS3, it is known that a replacement did not occur at DS3 when the first test symbol pool was introduced, meaning that the data symbol at DS3 cannot be data symbol 420-2 or 420-4. It is also known that a replacement took place when the second test symbol pool was introduced, meaning that the data symbol at DS3 must be data symbol 420-3 or 420-4. However, because data symbol 420-4 was previously eliminated from the first test symbol pool run, it is concluded that data symbol 420-3 is at DS3. With respect to DS4, it is known that no replacement occurred from either test symbol pool, meaning the data symbol at DS4 cannot be data symbol 420-2, 420-3 or 420-4. Thus, by process of elimination, the data symbol at DS4 must be 420-1. This logic and process of elimination process carries on for each of DS1-DS8 such that all data symbols at all data symbol locations are identified and the data storage gene 400 is read.

While FIGS. 4A and 4B provides a relatively simplified version of the reading step based on a small set of possible data symbols (four possible data symbol total), the same process can be used regardless of the number of possible data symbols in the DNA storage gene. In instance where a larger number of possible data symbols may be present in the DNA storage gene, it may be necessary to use more test symbol pools of differing compositions to collect the information required to make the logic/process of elimination analysis to identify the data symbols at each location of the DNA storage gene.

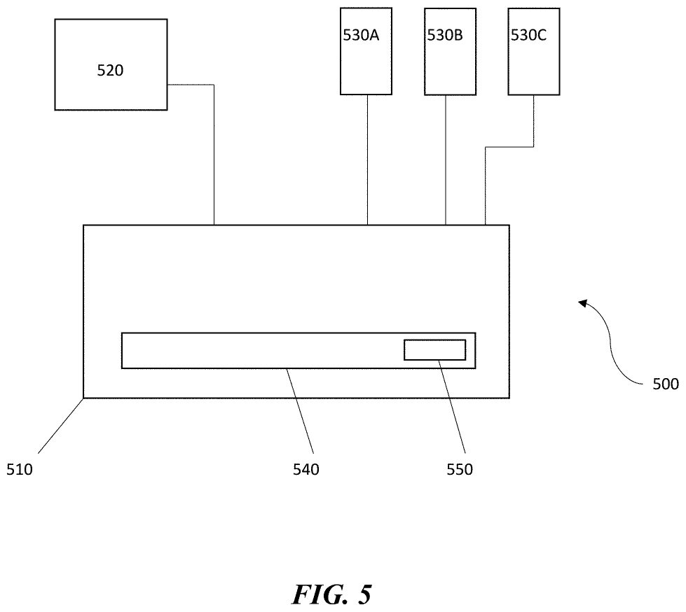

With reference to FIG. 5, a schematic illustration of a system 500 suitable for use in reading DNA storage genes includes a reaction vessel 510, an enzyme source 520, one or more test symbol pool sources 530A, 530B, 530C, a scanner 540 and a recorder 550. The reaction vessel 510 is generally configured to receive a DNA storage gene and other materials used in the process of reading the DNA storage gene. Any suitable type of reaction vessel can be used, and the reaction vessel can be of any size necessary for carrying out the reading of the DNA storage gene. The enzyme source 520 is in fluid communication with the reaction vessel 510 such that material contained within the enzyme source 520 can be supplied to the reaction vessel 510, such as via any suitable tubing or piping between the enzyme source 520 and the reaction vessel 510. The enzyme source 520 stores enzyme configured to remove one or more linking symbols from a DNA storage gene, such that when enzyme is supplied to the reaction vessel 510 from the enzyme source 520, the enzyme manipulates the DNA storage gene contained within the reaction vessel 510 so as to remove one or more linking symbols from the DNA storage gene. Any vessel suitable for storing enzymes and supplying enzymes to the reaction vessel 510 can be used for enzyme source 520.

System 500 further includes one or more test symbol pool sources 530A, 530B, 530C, with each test symbol pool source 530A, 530B, 530C being in fluid communication with the reaction vessel 510 via tubing or piping or the like such that test symbol pools stored within the test symbol pool sources 530A, 530B, 530C can be delivered into the reaction vessel 510. While FIG. 5 illustrates each test symbol pool source 530A, 530B, 530C having its own fluid connection with the reaction vessel 510, embodiments of the system 500 may also use a single fluid communication to fluidly connect all test symbol pool sources 530A, 530B, 530C with the reaction vessel 510. Similarly, while FIG. 5 shows the system 500 including three test symbol pool sources 530A, 530B, 530C, it should be appreciated that any number of test symbol pool sources may be included as part of the system 500. Each test symbol pool source 530A, 530B, 530C stores test symbol pools comprising a plurality of single stranded test symbols, with each test symbol comprising a data symbol and a linking symbol. In some embodiments, each test symbol pool source that forms part of the system 500 stores a different composition test symbol pool. Any vessel suitable for storing test symbol pools and supplying test symbol pools to the reaction vessel 510 can be used for test symbol pool sources 530A, 530B, 530C.

The system 500 further includes a scanner 540 that is configured to scan the DNA storage gene located within the reaction vessel 510. More specifically, the scanner 540 is configured to scan the DNA storage gene and distinguish between single stranded and double stranded DNA in the DNA storage gene, detect a single stranded DNA overhang, or both. The scanner 540 may be positioned within the reaction vessel 510 as shown in FIG. 5, though the scanner 540 may also be moveable in to and out of the reaction vessel 510 as necessary. As shown in FIG. 5, the system 500 may further include a recorder 550 configured to record each location on the DNA storage gene where the scanner 540 identified double stranded DNA or a single stranded DNA overhang, or both. As shown in FIG. 5, the recorder 550 may be part of or incorporated into the scanner 540, though the recorder 550 may also be a separate component located inside or outside of the reaction vessel 510, provided that the scanner 540 is able to communicate data to the recorder 550.

The above specification, examples, and data provide a complete description of the structure and use of example embodiments of the disclosed technology. Since many embodiments of the disclosed technology can be made without departing from the spirit and scope of the disclosed technology, the disclosed technology resides in the claims hereinafter appended. Furthermore, structural features of the different embodiments may be combined in yet another embodiment without departing from the recited claims.

I/We claim:

1. A method for use in reading a DNA storage gene, comprising:
(1) removing one or more linking symbols from a first strand of a DNA storage gene;
(2) introducing a test symbol pool to the DNA storage gene, the test symbol pool comprising:
a plurality of single stranded test symbols, each single stranded test symbol comprising a data symbol and a linking symbol;
(3) replacing a data symbol in the first strand of the DNA storage gene with a single stranded test symbol from the test symbol pool when the data symbol and the linking symbol in the single stranded test symbol are complimentary, respectively, to an adjacent linking symbol and data symbol in the second strand of the DNA storage gene;
(4) scanning the DNA storage gene to identify whether each linking symbol in the DNA storage gene is single stranded DNA or double stranded DNA; and
(5) recording each location on the DNA storage gene where the linking symbol is double stranded DNA.

2. The method of claim 1, wherein the test symbol pool is a first test symbol pool and includes a set of one or more different data symbols and each data symbol included in the first test symbol pool is known.

3. The method of claim 2, wherein steps (2)-(5) are repeated using a second test symbol pool including a set of one or more different data symbols, wherein each data symbol included in the second test symbol pool is known and wherein the set of data symbols in the second test symbol pool is different from the set of data symbols in the second test symbol pool.

4. The method of claim 3, wherein steps (2)-(5) using the second test symbol pool is carried out concurrently with steps (2)-(5) using the first test symbol pool.

5. The method of claim 3, wherein a combination of the recorded linking symbol locations and the known data symbols in each test pool is used to read the DNA storage gene.

6. The method of claim 1, wherein all linking symbols are removed from the first strand of the DNA storage gene.

7. The method of claim 1, wherein replacing data symbols in the first strand of the DNA storage gene with single stranded test symbols is carried out via toe-hold mediated strand displacement

8. The method of claim 1, wherein the linking symbol in each single stranded test symbol is positioned at an end of the single stranded test symbol.

9. The method of claim 1, wherein removing one or more linking symbols from the first strand of the DNA storage gene comprises:
introducing an enzyme to the DNA storage gene, the enzyme being programmed to cut the DNA storage gene at both ends of one or more linking symbols in the DNA storage gene; and
heating the DNA storage gene to remove the one or more linking symbols from the first stand of the DNA storage gene.

10. The method of claim 1, wherein each single stranded test symbol further comprises an anchor symbol and wherein the anchor symbol is located at one end of the single stranded test symbol and the linking symbol is located at an opposite or same end of the single stranded test symbol.

11. A method for use in reading a DNA storage gene, comprising:
(1) providing a first DNA storage gene having one or more linking symbols removed from a first strand of the DNA storage gene;
(2) introducing a first test symbol pool to the first DNA storage gene, the first test symbol pool comprising:
a plurality of single stranded test symbols, each single stranded test symbol comprising one of a first set of data symbols and one of a first set of linking symbols;
(3) replacing a data symbol in the first strand of the first DNA storage gene with a single stranded test symbol from the first test symbol pool when the data symbol and the linking symbol in the single stranded test symbol are complimentary, respectively, to an adjacent linking symbol and data symbol in the second strand of the first DNA storage gene;
(4) scanning the first DNA storage gene to identify whether each linking symbol in the first DNA storage gene is single stranded DNA or double stranded DNA;
(5) recording each location on the first DNA storage gene where the linking symbol is double stranded DNA;
(6) providing a second DNA storage gene having one or more linking symbols removed from a first strand of the second DNA storage gene, the second DNA storage gene being identical to the first DNA storage gene;
(7) introducing a second test symbol pool to the second DNA storage gene, the second test symbol pool comprising:
a plurality of single stranded test symbols, each single stranded test symbol comprising one of a second set of data symbols and one of the first set of linking symbols, the second set of data symbols being different from the first set of data symbols;
(8) replacing a data symbol in the second strand of the second DNA storage gene with a single stranded test symbol from the second test symbol pool when the data symbol and the linking symbol in the single stranded test symbol are complimentary, respectively, to an adjacent linking symbol and data symbol in the second strand of the second DNA storage gene;
(9) scanning the second DNA storage gene to identify whether each linking symbol in the second DNA storage gene is single stranded DNA or double stranded DNA;
(10) recording each location on the second DNA storage gene where the linking symbol is double stranded DNA; and
(11) using the recoded locations from steps (5) and (10) and the composition of the first test symbol pool and the second test symbol pool to read the DNA storage gene.

12. The method of claim 11, wherein all linking symbols are removed from the first strand of the first DNA storage gene and the second DNA storage gene.

13. The method of claim 11, wherein all data symbols and linking symbols in the first test symbol pool and the second test symbol pool are known.

14. The method of claim 11, wherein replacing data symbols in the first strand of the first DNA storage gene with single stranded test symbols and replacing data symbols in the first strand of the second DNA storage gene with single stranded test symbol is carried out via toe-hold mediated strand displacement

15. The method of claim 11, wherein the linking symbol in each single stranded test symbol is positioned at an end of the single stranded test symbol.

16. The method of claim 1, wherein removing one or more linking symbols from the first strand of the first DNA storage gene and from the first strand of the second DNA storage gene comprises:
introducing an enzyme to the first and second DNA storage gene, the enzyme being programmed to cut the first and second DNA storage gene at both ends of one or more linking symbols in the first and second DNA storage gene; and
heating the first and second DNA storage gene to remove the one or more linking symbols from the first stand of the first and second DNA storage gene.

17. The method of claim 11, wherein steps (1)-(5) are carried out concurrently with steps (6)-(10).

18. A system for use in reading a DNA storage gene, comprising:
a reaction vessel configured to receive a DNA storage gene;
an enzyme source in fluid communication with the reaction vessel, the enzyme source including an enzyme configured to remove one or more linking symbols from a first strand of a DNA storage gene;
one or more test symbol pool sources, each of the one or more test symbol pool sources being in fluid communication with the reaction vessel, each of the one or more test symbol pool sources comprising a test symbol pool comprising a plurality of single stranded test symbols, each single stranded test symbol comprising a data symbol and a linking symbol; and
a scanner configured to scan a DNA storage gene located in the reaction vessel and distinguish between single stranded DNA and double stranded DNA in the DNA storage gene, detect a single stranded DNA overhang, or both.

19. The system of claim 18, further comprising:
a recorder configured to record each location on the DNA storage gene where the scanner identifies double stranded DNA or a single stranded DNA overhang.

20. The system of claim 19, wherein the recorder is incorporated into the scanner.
